(19) United States
(12) Patent Application Publication
(10) Pub. No.: US 2024/0062907 A1
(43) Pub. Date: Feb. 22, 2024

(54) PREDICTING AN ANIMAL HEALTH RESULT FROM LABORATORY TEST MONITORING

(71) Applicant: Laboratory Corporation of America Holdings, Burlington, NC (US)

(72) Inventors: Ahmed Tawfik, Burlington, NC (US); Andy Newell, Burlington, NC (US)

(73) Assignee: Laboratory Corporation of America Holdings, Burlington, NC (US)

(21) Appl. No.: 18/451,730

(22) Filed: Aug. 17, 2023

Related U.S. Application Data
(60) Provisional application No. 63/398,942, filed on Aug. 18, 2022.

(51) Int. Cl. G16H 50/20; G16H 10/60; G16H 10/40

(52) U.S. Cl. CPC G16H 50/20; G16H 10/60; G16H 10/40

(57) ABSTRACT

The present disclosure relates to techniques for using machine-learning models to predict an animal health result from laboratory test monitoring. Particularly, aspects are directed to obtaining sets of data for a plurality of animal subjects over a time period. The sets of data include clinical observation data, body weight measurement data, outcome status data, or any combination thereof. The sets of data are processed into a training set of numerical values. A machine-learning model is trained on the training set to predict whether health of an animal subject is normal, veterinary attention for the animal subject is likely required in an upcoming time period, or an unplanned death outcome for the animal subject is likely in an upcoming time period. The machine-learning model is output.

### CROSS-REFERENCE TO RELATED APPLICATIONS

The present application claims priority and benefit from U.S. Provisional Application No. 63/398,942, filed Aug. 18, 2022, the entire contents of which are incorporated by reference herein for all purposes.

### FIELD

The present disclosure relates to clinical animal testing, and in particular to techniques for using machine-learning models to predict health results for an animal from their historical clinical laboratory testing data.

### BACKGROUND

Animals are often used for testing drugs before the drugs are made available for human use. In animal testing, clinical laboratories are healthcare facilities providing a wide range of laboratory procedures which aid veterinary technicians in carrying out the diagnosis, treatment, and management of animal subjects. Clinical laboratories report most laboratory test results or other examination results as individual numerical or categorical values. However, individual results, viewed in isolation, are typically of limited diagnostic value. To adequately use the results for animal subject diagnosis and management, clinicians usually must integrate many individual results from an animal subject and interpret them in the context of clinical data and medical knowledge, judgment, and experience. While this manual approach to data interpretation is the current standard in most cases, computational approaches to laboratory data integration and analysis offer tremendous potential to enhance diagnostic value. In particular, many animal subjects will have hundreds or thousands of these individual test results, often spanning years. As a consequence, many clinicians can easily overlook key results or important patterns and trends within sets of laboratory data. Furthermore, important diagnostic information may sometimes be contained within patterns across numerous data elements that may be too subtle or complex to identify without the aid of computational approaches. In addition, because the human brain faces great challenges in simultaneously considering a large number of data points, even the most experienced clinicians may be unable to extract all the useful information from existing clinical and laboratory data. To address these limitations and problems, machine-learning based approaches are disclosed herein that offer an opportunity to combine knowledge discovery with knowledge application to provide decision support based on previously unknown patterns. The use of machine-learning models to predict health results for an animal from historical clinical laboratory testing data improves data analysis and provides for better animal care.

### SUMMARY

In various embodiments, a computer-implemented method is provided that comprises: obtaining sets of data for a plurality of animal subjects over a time period, wherein the sets of data comprise: (i) clinical observation data, (ii) body weight measurement data, (iii) outcome status data, (iv) veterinary treatment record data, or (v) any combination thereof; processing the sets of data into a training set of numerical values; training a machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and outputting the machine-learning model.

In some embodiments, processing the sets of data into the training set of numerical values comprises: determining a free text entry in the sets of data; applying an embedding model to the free text entry to generate a vector of the free text entry; reducing a size of the vector using a principal component analysis reduction method; and including the vector in the training set.

In some embodiments, processing the sets of data into the training set of numerical values comprises: determining a categorical variable entry in the sets of data; converting the categorical variable entry into a numerical value using a mapping between numerical values and categorical variable entries; and including the numerical value in the training set.

In some embodiments, the method further comprises, prior to training the machine-learning model: labelling the numerical values of the training set with an unplanned death indicator, a veterinary request indicator, or a veterinary treatment indicator.

In some embodiments, labelling the numerical values of the training set comprises: determining the clinical observation data for an animal subject of the plurality of animal subjects includes the veterinary request indicator; and labelling the training set with the veterinary request indicator for the animal subject.

In some embodiments, labelling the numerical values of the training set comprises: determining the outcome status data for an animal subject of the plurality of animal subjects includes the unplanned death indicator; and labelling the training set with the unplanned death indicator for the animal subject.

In some embodiments, labelling the numerical values of the training set comprises: determining the veterinary treatment record data for an animal subject of the plurality of animal subjects includes the veterinary treatment indicator; and labelling the training set with the veterinary treatment indicator for the animal subject.

In some embodiments, training the machine-learning model comprises: generating a first decision tree based on values for the clinical observation data, the body weight measurement data, the veterinary treatment record data, and the outcome status data; determining an error associated with the first decision tree; and generating a second decision tree based on the error and the values for the clinical observation data, the body weight measurement data, the veterinary treatment record data, and the outcome status data, wherein the machine-learning model includes the first decision tree and the second decision tree.

In some embodiments, training the machine-learning model comprises: generating a chronologically ordered temporal graph for each animal subject based on the values for the clinical observation data, the body weight measurements data, the veterinary treatment record data, and the outcome status data; transforming the chronologically ordered temporal graph to a pre-processed table for classification; and automatically adjusting weights based on a predetermined condition.

In some embodiments, the machine-learning model comprises an additive gradient boosting algorithm or a multi-layer graph neural network algorithm.

In various embodiments, a computer-implemented method is provided that comprises: obtaining a set of data for an animal subject over a time period, the set of data including: (i) clinical observation data, (ii) body weight measurement data, (iii) outcome status data, (iv) veterinary treatment record data, or (v) any combination thereof; inputting the set of data into a machine-learning model trained for predicting a result for the animal subject, wherein the result comprises a veterinary request in an upcoming time period, an unplanned death outcome for the animal subject, or a treatment is likely to be administered to the animal subject in an upcoming time period; predicting, using the machine-learning model, the result for the animal subject; and outputting a classification based on the result for the animal subject.

In some embodiments, the machine-learning model is an additive gradient boosting algorithm or a multi-layer graph neural network algorithm.

In some embodiments, the classification comprises comparing the result for the animal subject to a determined threshold and classifying the animal subject as having the veterinary request in the upcoming time period, the unplanned death outcome for the animal subject is likely in an upcoming time period, or a treatment to the animal subject to be administered in an upcoming time period based on the comparison.

In some embodiments, the computer-implement method further comprises providing a recommendation based on the classification of the animal subject.

In some embodiments, the computer-implement method further comprises providing the classification and/or the recommendation to a user through a graphical user interface (GUI).

In some embodiments, the computer-implement method further comprises, prior to receiving the set of data for the animal subject: obtaining sets of data for a plurality of animal subjects over a time period, wherein the sets of data comprise the clinical observation data, the body weight measurement data, the outcome status data, the veterinary treatment record data, or any combination thereof; processing the sets of data into a training set of numerical values; training the machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and outputting the machine-learning model.

In some embodiments, processing the sets of data into the training set of numerical values comprises: determining a free text entry in the sets of data; applying an embedding model to the free text entry to generate a vector of the free text entry; reducing a size of the vector using a principal component analysis reduction method; and including the vector in the training set.

In some embodiments, processing the sets of data into the training set of numerical values comprises: determining a categorical variable entry in the sets of data; converting the categorical variable entry into a numerical value using a mapping between numerical values and categorical variable entries; and including the numerical value in the training set.

In some embodiments, the computer-implement method further comprises, prior to training the machine-learning model: labelling the numerical values of the training set with an unplanned death indicator, a veterinary request indicator, or a veterinary treatment indicator.

In some embodiments, labelling the numerical values of the training set comprises: determining the clinical observation data for an animal subject of the plurality of animal subjects includes the veterinary request indicator; and labelling the training set with the veterinary request indicator for the animal subject.

In some embodiments, labelling the numerical values of the training set comprises: determining the outcome status data for an animal subject of the plurality of animal subjects includes the unplanned death indicator; and labelling the training set with the unplanned death indicator for the animal subject.

In some embodiments, labelling the numerical values of the training set comprises: determining the veterinary treatment record data for an animal subject of the plurality of animal subjects includes the veterinary treatment indicator; and labelling the training set with the veterinary treatment indicator for the animal subject.

In some embodiments, training the machine-learning model comprises: generating a first decision tree based on values for the clinical observation data, the body weight measurement data, the veterinary treatment record data, and the outcome status data; determining an error associated with the first decision tree; and generating a second decision tree based on the error and the values for the clinical observation data, the body weight measurement data, the veterinary treatment record data, and the outcome status data, wherein the machine-learning model includes the first decision tree and the second decision tree.

In some embodiments, training the machine-learning model comprises: generating a chronologically ordered temporal graph for each animal subject based on the values for the clinical observation data, the body weight measurements data, the veterinary treatment record data, and the outcome status data; transforming the chronologically ordered temporal graph to a pre-processed table for classification; and automatically adjusting weights based on a predetermined condition.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1A depicts a block diagram illustrating a machine learning system for training and deploying machine-learning models in accordance with various embodiments.

FIG. 1B shows an example of a temporal, forward-in-time graph in accordance with various embodiments.

FIG. 2 shows a flowchart illustrating a process for training a machine-learning model according to various embodiments.

FIG. 3 shows a flowchart illustrating a process for using a machine-learning model to predict an animal health result according to various embodiments.

FIG. 4A shows an exemplary graphical user interface (GUI) that displays animal species according to various embodiments.

FIG. 4B shows an exemplary secondary user interface (UI) that displays animal health scores according to various embodiments.

FIG. 5 shows an example of a computing environment to perform the disclosed techniques according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

I. Introduction

Machine learning (ML) has had tremendous impacts on numerous areas of modern society. For example, it is used for filtering spam messages from text documents, such as e-mail, analyzing various images to distinguish differences, and extraction of important data from large datasets through data mining. ML makes it possible to uncover patterns, construct models, and make predictions by learning from training data. ML algorithms are used in a broad range of domains, including biology and genomics. Deep learning (DL) is a subset of ML that differs from other ML processes in many ways. Most ML models perform well due to their custom-designed representation and input features. Using the input data generated through that process, ML learns algorithms, optimizes the weights of each feature, and optimizes the final prediction. DL attempts to learn multiple levels of representation using a hierarchy of multiple layers. In recent years, DL has overtaken ML in many areas, including speech, vision, and natural language processing. DL and ML are also increasingly used in the medical field, mainly in the areas of image analysis, drug research and development, data mining from medical documents, and speech. In addition to image and text data from medical charts generated in hospitals, various types of laboratory data may also be analyzed, which are mostly composed of numbers assigned various units of measurement. However, very few DL and/or ML models have been developed to analyze laboratory data for animal subjects involved in drug trials.

In practice, physical examinations performed by veterinarians and laboratory test results are generally needed to evaluate an animal subject's status and predict an outcome. Electronic clinical decision support represents an important tool to improve evaluation of these various clinical data sets and the efficiency with which data can be converted into useful information. The main purpose of clinical decision support is to provide timely information to veterinarians, clinicians, and others to inform decisions about health care. Examples of clinical decision support tools include order sets created for particular conditions or types of animal subjects, recommendations, and databases that can provide information relevant to particular animal subjects, reminders for preventive care, and alerts about potentially dangerous situations. Rule-based algorithms provide the foundation for most conventional clinical decision support tools. Rule-based algorithms tend to be easier to develop, validate, implement, and explain and can often be adapted directly from guidelines or literature. However, rule-based algorithms applied in clinical practice provide decision support based on previously established knowledge.

To address these limitations and problems, machine-learning based approaches are disclosed herein that offer an opportunity to combine knowledge discovery with knowledge application to provide decision support based on previously unknown patterns. These previously unknown patterns are discovered and implemented to make inferences with respect to clinical decision support (e.g., recommendation of a veterinary visit to inform a technician of a possible disease state for a patient) using machine-learning models. In an illustrative embodiment, a method is provided that comprises: obtaining sets of data for a plurality of animal subjects over a time period, wherein the sets of data comprise: (i) clinical observation data, (ii) body weight measurement data, (iii) outcome status data, (iv) veterinary treatment record data, or (v) any combination thereof; processing the sets of data into a training set of numerical values; training a machine-learning model on the training set of numerical values to predict whether health of an animal subject is normal, veterinary attention for the animal subject is likely (i.e., a high probability) required in an upcoming time period, an unplanned death outcome for the animal subject is likely in an upcoming time period, or a treatment is likely to be administered to the animal subject in an upcoming time period; and outputting the machine-learning model.

In another illustrative embodiment, a method is provided that comprises: obtaining a set of data for an animal subject over a time period, the set of data including: (i) clinical observation data, (ii) body weight measurement data, (iii) outcome status data, (iv) veterinary treatment record data, or (v) any combination thereof; inputting the set of data into a machine-learning model trained for predicting a result for the animal subject, wherein the result comprises a veterinary request in an upcoming time period, an unplanned death outcome for the animal subject, or a treatment administered to the animal subject in an upcoming time period; predicting, using the machine-learning model, the result for the animal subject; and outputting a classification based on the result for the animal subject.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something. As used herein, the term “result” or “animal health result” means a value determined as a result of analyzing a single type of clinical data (e.g., clinical observation data, body weight measurement data, veterinary treatment record data, or outcome status data) or a score determined as a result of analyzing one or more types of clinical data. In some instances, the value is numerical such as 1.0, 2.5, 19.7, 25.0, etc. In other instances, the value is alpha such as negative, abnormal, positive, normal, etc. In other instances, the value is alpha numeric such as abnormal between 2.0 and 5.0 or the like.

II. Animal Health Result Prediction Models and Techniques

In various embodiments, machine-learning models and techniques are provided that predict an animal health based on animal health history to accurately identify animal subjects trending towards veterinary assistance or an unplanned death and to support veterinarians and healthcare organizations by potentially informing diagnosis early. Conventionally, animal health results are predicted manually by veterinarians or other operational staff analyzing health data for animal subjects. When many animal subjects are being cared for and assessed at once, the veterinarians and operational staff may be analyzing large amounts of data. Accordingly, subtle changes in animal health may be difficult to detect and, if the changes are caught, they may be caught when it is too late to intervene to improve the health of an animal subject.

In order to overcome this challenge and others, the techniques described herein use a machine-learning model to predict results for animal subjects. The machine-learning model can promote animal health and well-being by predicting negative outcomes (e.g., veterinary request, unplanned death, or administration of a treatment) and notifying an appropriate entity prior to the negative outcome occurring for an animal subject. The machine-learning model can be a classification model that uses an ensemble, or weak learner, approach, or the machine-learning model can be a classification model that uses graph neural networks. Data may be received in various formats that can be converted into numerical values that are usable by the machine-learning model. For example, a word embedding model may be used to convert free text entries into a vector of a numerical value that represent a semantic meaning of the free text. In addition, categorical variables, which can be predefined, may be mapped to numerical values. So, the mapping can be used to convert a particular categorical variable into its corresponding numerical value.

In machine learning, ensembles combine multiple hypotheses to form a more suitable hypothesis that makes accurate predictions. Ensemble learning combines several base algorithms to form one optimized predictive algorithm. For example, a typical decision tree for classification takes several factors, turns them into rule questions, and given each factor, either makes a decision or considers another factor. The result of the decision tree can become ambiguous if there are multiple decision rules, e.g., if a threshold to make a decision is unclear or new sub-factors are input for consideration. This is where ensemble methods can help to form a more suitable hypothesis. Instead of being reliant on one decision tree to make the right call or be accurate, ensemble methods take several different trees and aggregate them into one final more suitable hypothesis that operates as a strong predictor.

In some embodiments, the machine-learning models implement a boosting technique as the ensemble method. The boosting algorithm tries to build a strong learner (predictive model) from the mistakes of several weaker models. Boosting makes ‘n’ number of models during the model training period. Initially, boosting starts by creating a first model (e.g., a decision tree) from the training data. As the first model is made and errors from the first model are noted by the boosting algorithm, the sample or record which is incorrectly classified is used as input for a subsequent model. The subsequent model is generated from the previous model (e.g., the first model) by trying to reduce the errors from the previous model. Models are added sequentially, each correcting its predecessor, until the training data is predicted in accordance with an acceptance criterion (e.g., >90% accuracy) or a maximum number of models have been added to the ensemble. Essentially, the boosting tries to reduce the bias error which arises when models are not able to identify relevant trends in the data. This happens by evaluating a difference between the predicted value of the model and the actual value or ground truth value assigned the training data. There are various types of boosting that may be implemented such as adaptive boosting (AdaBoost), gradient tree boosting, or XGBoost.

In certain instances, the machine-learning model implements an additive gradient boosting technique. Additive gradient boosting combines multiple weak classifiers to build one strong classifier. A weak classifier is one that performs better than random guessing, but still performs poorly at designating classes to objects. A single weak classifier may not be able to accurately predict the class of an object, but when multiple weak classifiers are grouped with each one progressively learning from the others' wrongly classified objects, a single strong model can be generated. The classifier could be any classifier such as a decision tree, logistic regression, or the like. Generating the single strong model may be implemented via a training process comprising generating a weak classifier (e.g., a decision tree) using training data based on weighted samples (e.g., animal health results). The weights of each sample indicate how important it is to be correctly classified. Initially, for the first model, all the samples may have equal weights. A weak classifier for each variable may be generated and a determination may be made as to how well each weak classifier classifies samples to their target classes. For example, a first subset of animal subjects may be evaluated and a determination is made as to how many samples are correctly or incorrectly classified as involving a veterinary request or an unplanned death for each weak classifier. Based on the determination, an error of the determination can be calculated. The next weak classifier can then be generated based on the error (e.g., a gradient of the error) to improve the classification in the next weak classifier. Thereafter the training process is reiterated until all the samples have been correctly classified, or a maximum iteration level has been reached.

Artificial neural networks such as multi-layer neural networks can also be used in machine learning to make accurate prediction. These networks learn by using many examples of input data (referred to as features or variables) along with output data. The objective is to find an optimal function that transforms the input into the output effectively and accurately. The basic unit in neural networks is the neuron, which may be represented as a linear equation (w*x+b)., where the slope (w) and intercept (b) are known as learnable parameters. An essential step in neural networks is the introduction of an activation function, a differentiable and non-linear function. This function modifies the linear equation's output, allowing each neuron to adopt more intricate structures. These neurons collectively form layers within the neural network. The network's capacity to comprehend complex, non-linear relationships between input and output data increases with the addition of more layers. In the case of deep neural networks, which can comprise tens to hundreds of layers, the term “deep” is derived.

The neural network's training involves multiple iterations over the same dataset. During these iterations, the learnable parameters are updated progressively, aiming to minimize the difference between the network's current predictions and the actual expected outputs. This iterative parameter update procedure is known as gradient descent. Due to the multiple passes over the same data, neural networks can start memorizing the output specifics rather than grasping a generalized function. To tackle this, a portion of the data is reserved during training as a representative subset. This withheld portion helps compute a validation error, which is evaluated against the training errors. A clear indication of overfitting arises when the hold-out error isn't diminishing with updates, while the learned error is decreasing after each update.

A well-performing model is usually identified during initial iterations, where both the hold-out error and the learned error are relatively small and comparable to previous iterations. This suggests that the model is effectively learning without falling into the trap of overfitting.

In some embodiments, the machine-learning models implement a multi-layer graph neural network technique as the neural network method. The multi-layer graph neural network can capture known relationships between input data examples by constructing a graph. Input data from connected examples enables information to be shared (aggregated) before undergoing the typical gradient descent learning process of traditional neural networks, a process known as message passing. The number of times message passing occurs can be specified but can lead to oversmoothing of information if done excessively. This information sharing across examples enables domain knowledge of relations to guide the learning process, often leading to a better model. For instance, if the data is collected sequentially over time, a simple sequential connection between observations can be used to create a directed forward-in-time graph. This approach would provide a more accurate description of the data than treating the data as independent, as is often the case in typical neural networks. Various types of graph neural networks exist, differing in the method used for aggregating information across connected observations. Examples include the graph convolutional neural network (GCN) and the graph attention network (GAT).

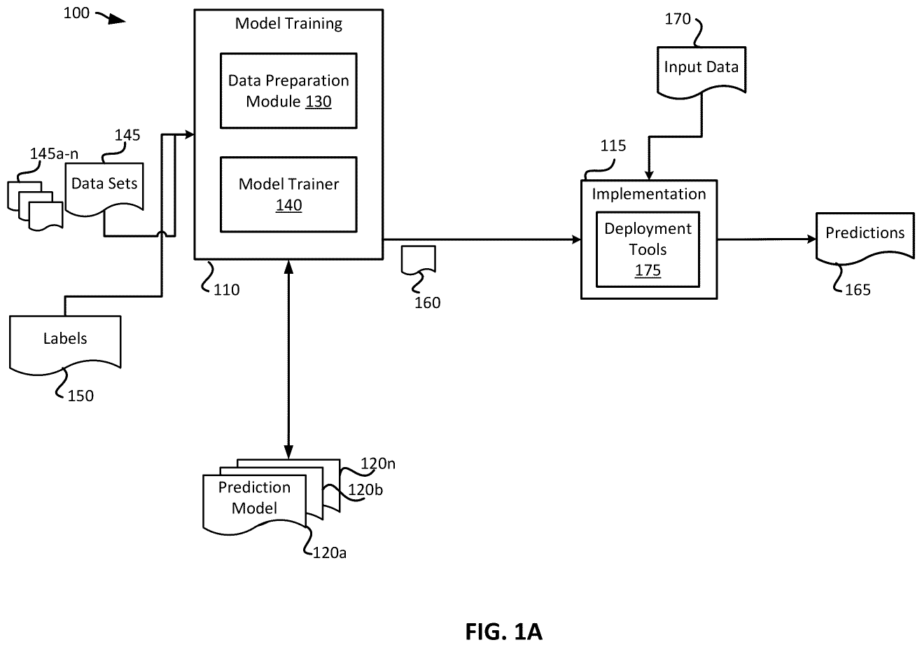

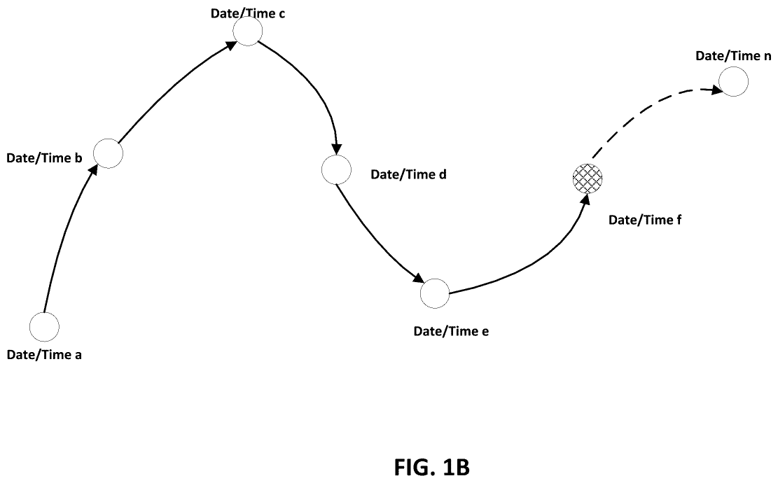

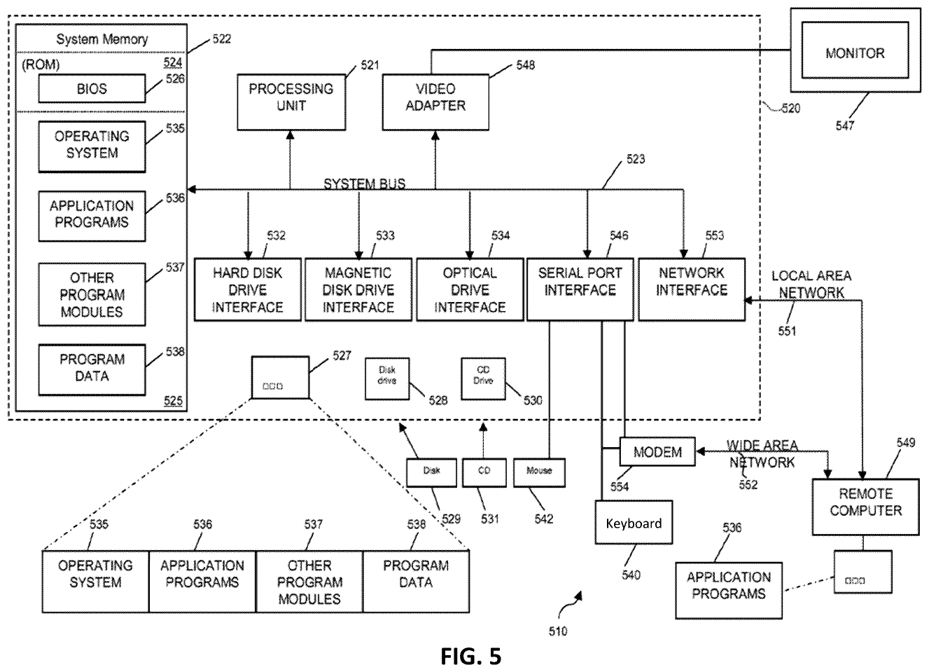

FIG. 1A is a block diagram illustrating a machine learning system 100 in accordance with various embodiments. As shown in FIG. 1A, the machine learning system 100 includes various subsystems or services: a prediction model training subsystem or service 110 to build and train models and an implementation subsystem or service 115 for implementing one or more models using a computing system (e.g., computing environment 510 in FIG. 5). The prediction model training subsystem or service 110 builds and trains one or more prediction models 120a-120n (‘n’ represents any natural number) to be used by the other subsystems or services (which may be referred to herein individually as a prediction model 120 or collectively as the prediction models 120). For example, the prediction models 120 can include a model for predicting a class (also referred to herein as a classification) for an animal subject, the class being identified from a plurality of target classes comprising: 0=a normal status for the animal subject, 1=veterinary attention for the animal subject is likely (i.e., a high probability) required in an upcoming time period 0.1 may also represent other classification of interest, for example, an unplanned death outcome for the animal subject is likely in an upcoming time period or a treatment is likely to be administered to the animal subject in an upcoming time period. In some instances, the prediction models 120 can provide multi-class classification, and use different number to represent different classes. For example, 2=an unplanned death outcome for the animal subject is likely in an upcoming time period, and 3=a treatment is likely to be administered to the animal subject in an upcoming time period. Still other types of prediction models may be implemented in other examples according to this disclosure.

A prediction model 120 can be a machine learning (“ML”) model, such as a convolutional neural network (“CNN”), e.g., an inception neural network, a residual neural network (“Resnet”), a recurrent neural network, e.g., long short-term memory (“LSTM”) models or gated recurrent units (“GRUs”) models, or a multi-layer graph neural network, e.g., a convolutional graph network (GCN), or other variants of Deep Neural Networks (“DNN”) (e.g., a multi-label n-binary DNN classifier or multi-class DNN classifier). A prediction model 120 can also be any other suitable ML model trained for providing a prediction, such as a Generalized linear model (GLM), Generalized additive model (GAM), Support Vector Machine, Bagging Models such as Random Forest Model, Boosting Models, Shallow Neural Networks, or combinations of one or more of such techniques—e.g., CNN-HMM or MCNN (Multi-Scale Convolutional Neural Network). The model can also be an ensemble of base models (e.g., decision trees or neural networks) combined via bagging, boosting, or stacking to create an optimal predictive model, e.g., a boosting model such as an AdaBoost or Gradient Boosting model. The machine learning system 100 may employ the same type of prediction model or different types of prediction models for providing predictions to users. In certain instances, the prediction model 120 performs predictions using an additive gradient boosting algorithm. Still other types of prediction models may be implemented in other examples according to this disclosure.

To train the various prediction models 120a-120n, the training subsystem or service 110 is comprised of two main components: data preparation module 130 and model trainer 140. The data preparation module 130 performs the processes of loading data sets 145, splitting the data sets 145 into training and validation sets 145a-n so that the system can train and test the prediction models 120, and pre-processing of data sets 145. The splitting the data sets 145 into training and validation sets 145a-n may be performed randomly (e.g., a 90/10% or 70/30%) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting.

The data sets 145 are acquired from a clinical laboratory or health care systems (e.g., animal subject record system, clinical trial testing system, and the like). In some instances, the data sets 145 are acquired from a data storage structure such as a database, a laboratory or hospital information system, or the like associated with the one or more modalities for acquiring health data for subjects. Additionally, or alternatively, the data preparation module 130 may standardize the format of the data. In certain instances, the data sets 145 comprise: (i) clinical observation data, (ii) body weight measurement data, and (iii) outcome status data. Clinical observation data can include health data of an animal subject generated by a physical examination of the animal subject. The clinical observation data may include fields with predefined and selectable or definable categorical variables. Body weight measurement data can include a measurement of a weight of an animal subject along with an indication of a time in which the measurement was taken. The outcome status data can include an indication of a viability or morbidity of the animal subject. The veterinary treatment record data can include historical health data of the animal subject generated during veterinary visits.

In some instances, the data sets 145 are stored or standardized by the data preparation module 130 to be stored in a data structure that is appropriate for training (e.g., a list, a graph, a table, a matrix, or the like). Table 1 provides an example of various clinical observation data. Table 2 provides an example of body weight measurement data. Table 3 provides an example of outcome status data. Table 4 provides an example of treatment table that specifies the time a given medication or treatment was given. The data structure used to store the data sets 145 may be prepared using a design based on any one or a combination of Tables 1-4. For example, the data structure is a matrix of size m×n×p, and with m rows storing data of m animal subjects, and each row corresponding to one animal subject. The n columns of the matrix may correspond to an ordered list of entries, with each entry corresponding to an item in one of the Tables 1-4. The data stored in each cell of the matrix is a value of the corresponded item taken at a specific time. For each item, the animal subject can have p different values storing in the p dimension of the matrix.

In some instances, the data sets 145 are stored in a data structure comprising adjacency graphs, adjacency tables and/or adjacency lists that are similar to the matrix described above. FIG. 1B shows an example of a temporal, forward-in-time graph in accordance with various embodiments. Each node in FIG. 1B represents a datum associated with an animal subject at a time. Different node patterns in FIG. 1B represents a different type of data. For example, a hollow circle may represent a clinical observation, and a cross pattern may represent a body weight measurement. In FIG. 1B there is a total of n observations where each node corresponds to an individual clinical observation, a body weight measurement, an outcome status, or a veterinary treatment record. Nodes are connected forward in time, meaning that each node can only propagate information about an observation to future observations. In some instances, the graph can be backward, bidirectional, or multidirectional.

In some instances, adjacency graphs and/or adjacency lists are used when the prediction model 120 is a multi-layer graph neural network (GNN) model. Using adjacency graphs (or adjacency tables, adjacency lists) in training the GNN offers advantages that contribute to the efficiency and effectiveness of the learning process. First, the adjacency matrix/table/list provides a compact and efficient way to represent the connectivity structure of a graph. It captures relationships between nodes in a concise format, which is crucial for scaling up to large graphs. Second, in many instances, the graphs for animal subjects are sparse, meaning that most nodes are not directly connected. The adjacency matrix/table/list efficiently encodes this sparsity, allowing GNN algorithms to focus computation only on relevant neighbors, reducing computational overhead. Additionally, the GNN relies on aggregating information from neighboring nodes to update a node's representation, and the adjacency matrix/table/list simplifies the process of identifying and accessing neighbors, enabling efficient message passing. Furthermore, the adjacency matrix/table/list can be exploited for parallelism during training. Operations like message aggregation and update can be parallelized across nodes, enhancing the overall training speed. In some instances, the graphs representing the training animal subjects are substantially larger involving millions of nodes and edges, and the adjacency matrix's efficient representation becomes increasingly important. GNNs can handle graphs with millions of nodes and edges by leveraging the sparsity and compactness of the adjacency matrix. Additionally, the adjacency matrix/table/list is not restricted to a specific graph type. It can be used for various graph structures, including directed and undirected graphs, as well as graphs with self-loops, allowing easy transformation of the graph by adding or removing edges, which is useful in scenarios where the graph evolves over time. Moreover, the adjacency matrix/table/list can be used for visualizing the graph's connectivity and relationships, aiding in understanding the graph's structure in a GUI.

While traditional machine learning algorithms make the assumption that each observation is independent, GNNs provide an explicit way to represent dependencies across observations. This can be particularly advantageous when dealing with temporal data that is clearly correlated over time, which is the case when tracking an animal subject's health. GNNs also allow for the injection of domain knowledge and this domain knowledge can then be formalized in the graph structure itself. For example, experienced veterinary technicians might suggest that because food consumption observations are made daily then it might make sense to connect observations related to an animal subject's food consumption over time directly in the graph despite there being other observations made in between. This would allow the GNN to learn from both the temporal dependency and knowledge about the type of observations. Traditional machine learning algorithms do not have this capability and would rely on individual examples to learn such a dependency.

TABLE 1

| SITE_NAME | Site_1 |
|---|---|
| MEASUREMENT_NAME | Large |
| | Animal |
| CATEGORY_NAME | Skin |
| | Observation |
| SUBCATEGORY_NAME | Scarring |
| MODIFIER_1 | Redness |
| MODIFIER_2 | Minor |
| | bleeding |
| MODIFIER_3 | Dryness |
| MODIFIER_4 | Bumps |
| MODIFIER_5 | discoloration |
| LOCATION_MODIFIER | arms |
| SPECIES_NAME | Monkey |
| DATE_TIME_TAKEN | Feb./4/2019 |
| | 12:30 |
| PRETEST_NUMBER | P1039420 |

TABLE 2

| SITE NAME | Site_1 |
|---|---|
| WEIGHT | 3100 |
| STORED_UNITS | g |
| SPECIES_NAME | Monkey |
| DATE_TIME_TAKEN | Dec./23/2019 |
| | 12:30 |
| PRETEST_NUMBER | P1039420 |

TABLE 3

| SITE_NAME | Site 1 |
|---|---|
| DEATH_CODE_NAME | Terminal |
| SPECIES_NAME | Rat |
| DATE_TIME_TAKEN | Jul./14/2019 |
| | 12:30 |
| PRETEST_NUMBER | P1039420 |

TABLE 4

| SITE_NAME | Site_1 |
|---|---|
| GIVEN | 1 |
| SPECIES_NAME | Rat |
| DATE_TIME_TAKEN | Jul./22/2020 |
| | 12:30 |
| PRETEST_NUMBER | P1300209 |

In some instances, data in the data sets 145 are collected and stored using the same process. Data can be collected by routine physical and visual inspection of the animal subjects and their living area, digital or analog measurement tools such as scales, clinical lab tests, clinical measurements such as neurological exams, clinical interventions such as treatment, or cataloging general outcomes. These routine inspections can be performed by trained staff (e.g., veterinaries, veterinary technicians, or other veterinary operations and animal specialists) that are capable of identifying irregularities in behavior, food consumption, appearance, and other clinical health indicators in animal subjects. Data are collected at a daily, sub-daily, or on-demand frequency by trained staff and typically stored in a relational database. In some instances, the observations made by the trained staff are entered into a database via a GUI where an entry is comprised a single observation made for a single animal subject. The database contains information about each animal subject and is delimited by a unique identifier (e.g., PRETEST_NUMBER in Table 1) such that trained staffed can enter clinical observations for that specific animal subject.

In some instances, the unique identifier also serves as the primary key for linking the various data tables. In some instances, each type of observation is placed into its own tabular data table where a row consists of an observation instance for a single animal subject at a particular time. In some instances, there are four data tables: Table 1 for clinical observations, Table 2 for body weight measurements, Table 3 for outcome data, and Table 4 for treatments administered. Other metadata regarding the species (SPECIES_NAME), animal sex (SEX), and site the animal subject is located (SITE_NAME) may also be recorded and included in each observational data entry. Each entry may also have a date and time stamp when an observation is made, for example, listed under DATE_TIME_TAKEN.

Data entered into the GUI can be done in several ways. For example, the data may be entered via a dropdown menu where only predefined fields can be selected (e.g., animal sex), a character-limited free text field where the trained staff can enter any comments within a character limit, date-time fields to store times, numeric fields, or Boolean fields.

In some instances, the data to be used as training data can also be collected from a historical database or a publicly available database. The data can be entered or stored in the data table or other suitable data structure using similar techniques as described above.

The training process for prediction model 120 may include preprocessing the data sets 145 to standardize the data sets 145 into numerical values interpretable by one or more algorithms to be trained as a prediction model 120. For instance, the data preparation module 130 may determine that the data sets 145 include a free text entry, or an entry that is definable and not selectable from a predefined list. For example, MODIFIER_1 in Table 1 is an example of a free text entry. Upon detecting the free text entry, the data preparation module 130 can apply an embedding model to the free text entry to generate a vector of the free text entry. For example, the embedding model may be Word2Vec, GloVe, or any other suitable word embedding model. The embedding model can be pre-trained on open-source biomedical and scientific corpuses to generate a vector of size 200 (or any other suitable size). The vector can represent the semantic meaning of the free text in a numerical value. The data preparation module 130 may then reduce a size of the vector so that the vector may be more easily used in downstream computations. For example, a principal component analysis reduction method may be used to reduce the vector from size 200 to size 20 (or any other suitable size). Reducing the size of the vector can allow the prediction model 120 to learn by drawing attention to only those vector components that contain the most information. Once the vector of the reduced size is generated, the vector can be included in a training data set.

The data preparation module 130 may additionally convert categorical variables to numerical values. For instance, the data preparation module 130 may determine that the data sets 145 include a categorical variable entry, or an entry that is predefined and selectable from a predefined list. As an example, the entry of “Large Animal” for MEASUREMENT NAME in Table 1 is a categorical variable entry that is convertible to a numerical value. Each possible categorical variable entry can be mapped to a predefined numerical value, which may be chosen arbitrarily. For example, the categorical variable entries for MEASUREMENT NAME may include “Large Animal”, “Vet Request”, “Neurological Exam”, etc. The mapping can associate “Large Animal” with a numerical value of one, “Vet Request” with a numerical value of two, “Neurological Exam” with a numerical value of three, etc. The mapping may be saved in a database accessible by the data preparation module 130. So, upon detecting a categorical variable entry, the data preparation module 130 can convert the categorical variable entry into a numerical value using the mapping and then including the numerical value in the training set.

In some instances, prior to training a machine-learning model, data is extracted and pre-processed into a data structure that is appropriate for training. The extraction process may involve on-demand querying the data tables (Table 1-4) from the database for all animal subjects using standard query language. In some instances, each table is still separate and will be pre-processed individually after the extraction. In some instances, the tables are combined or concatenated to be stored in a new table.

The pre-processing step may involve standardize the data or the data structure. In some instances, a text column that contains more than a predetermined amount of unique options is transformed into embedding vectors by using embedding methodologies, such as bag-of-words, Word2Vec, or large language model transformer model embeddings. For example, if a text column contains an open text field where the user can write anything within a character limit then a phrase such as “low food consumption” would map to an embedding vector that can represent its component words and/or semantic meaning. If there are fewer than the predetermined limit of unique options, the text field may be integer encoded, where each unique options is assigned a unique integer value. For example, if a text column contains animal sex then the unique options are two, “Male” and “Female”, and they can be encoded to zero and one, respectively.

In some instances, the table containing clinical observations is further pre-processed by removing any measurements that would imply an outcome. For example, if an exam that is only performed during detailed veterinary requests (a potential target outcome) is recorded in the clinical observation table, then it will be excluded during training to avoid information leakage.

In some instances, the body weight measurements (WEIGHT in Table 2) are normalized using one of the commonly-used normalization techniques prior to training, such as standard scalar normalization or minimum-maximum normalization. Once the clinical observations and body weight tables are pre-processed, they can be merged into a single table using the animal subject's unique identifier as the merge key. This combined table is then sorted by animal subject unique number and date-time in chronological order from oldest measurement to most recent. Body weight measurements may be presumed to be that same across time unless a new measurement is made.

Depending the particular embodiment of the prediction model, additional pre-processing steps may be required prior to training. For example, in the case of a graph neural network, a directed acyclic graph may be constructed for each animal subject from the merged clinical observations and body weight table, where each node represents a measurement in time.

Once the pre-processed and merged clinical observations and body weight tables (input features table) and the labels table are created they are stored on some sort of storage device with a tag that identifies when the data were created. From here the particular embodiment of the machine-learning algorithm can read these data from the storage device and the model can be trained to predict the target labels. The training process is triggered on-demand and requires the user to identify which tagged version of the training data to use.

The training data 145a for a prediction model 120 may include historical data and labels 150 corresponding to ground truths of a normal health, a veterinary request, an unplanned death outcome occurring for, or a treatment administered to animal subjects. The historical data comprises clinical observations and body weight measurements. An opened veterinary request represents a set of animal observations that were identified by veterinary technicians as concerning and therefore involving greater attention from the veterinarian and potentially resulting in a prescribed treatment plan for the animal subject. A veterinary request indicator may be included in the clinical observation table as a categorical variable entry (e.g., in the MEASUREMENT_NAME column). An unplanned death indicator may be included in the outcome status data as a categorical variable entry (e.g., in the DEATH_CODE_NAME column). A veterinary treatment indicator may be included in the outcome status data as a categorical variable entry (e.g., a Boolean variable in the GIVEN column). In some examples, for each data set, an indication of the correct result to be inferred by the prediction model 120 may be provided as ground truth information (e.g., the unplanned death indicator, the veterinary request indicator, or the veterinary treatment indicator) for labels 150. A normal health may be inferred by an absence of the unplanned death indicator, the veterinary request indicator or the veterinary treatment indicator. In some instances, the labels 150 may be obtained from a data structure used to maintain data consistency across training samples. The behavior of the prediction model 120 can then be adapted (e.g., through back-propagation) to minimize the difference between the generated inferences for various entities and the ground truth information.

In some instances, training labels comprises data extracted from multiple sources, including the clinical observations table (Table 1), outcome status table (Table 3), and the treatment table (Table 4). In some instances, veterinary requests, recorded within the clinical observations table, may represent a significant outcome category for prediction purposes. In such instances, veterinary requests may be identified within the clinical observations table, accompanied by relevant date-time stamps and essential animal metadata. Similarly, instances involving unplanned animal deaths from the outcome status table and administered treatments from the treatment table may be isolated, retaining their associated metadata and date-time details. These distinct data sets are subsequently amalgamated into a unified table, organized by unique animal subject identifiers and chronological date-time sequences. To facilitate supervised learning, in some instances, specific target outcomes are encoded as integers, each class being mapped to a distinct non-zero integer value. The integer value of zero is specifically reserved to denote time intervals during which an animal subject has not yet encountered any relevant outcomes. For instance, if an animal subject exhibits unremarkable observations without concurrent veterinary requests during a given week, its label for routine observations during that period is designated as zero. This consolidated encoded table serves as the pivotal training label for supervised learning endeavors.

In some instances, clinical observations can be made by veterinary technicians or other trained clinical experts as part of routine evaluation and monitoring. These observations can be conducted through visual inspections of individual animal subjects or through some form of group observation. For example, an individual observation might entail noticing that an animal subject appears to be thinning, while a group observation could involve discovering feces in a location where multiple animal subjects tend to gather, with uncertainty regarding the responsible animal subject. These clinical observations can be conducted on a daily or sub-daily basis and can be recorded using a computer-based system. Body weight measurements can also be taken by veterinary technicians or other trained clinical experts as part of routine evaluation and monitoring. These measurements can be obtained using standard digital and analog scales, and the weight can be recorded on a daily basis within a computer-based system.

The model trainer 140 performs the processes of determining hyperparameters for the prediction model 120 and performing iterative operations of inputting examples from the training data 145a into the prediction model 120 to find a set of model parameters (e.g., weights and/or biases) that minimizes a cost function(s) such as loss or error function for the prediction model 120. The model trainer 140 is part of a machine learning operationalization framework comprising hardware such as one or more processors (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for training the prediction model 120. In some instances, the model trainer 140 performs training using at least a GPU. The input data size is generally several gigabytes, and a GPU can provide better computing performance to further improve the computing cost and efficiency.

The hyperparameters are settings that can be tuned or optimized to control the behavior of the prediction model 120. Most models explicitly define hyperparameters that control different features of the models such as memory or cost of execution. However, additional hyperparameters may be defined to adapt the prediction model 120 to a specific scenario. For example, the hyperparameters may include the number of hidden units of a model, the learning rate of a model, the convolution kernel width, the number of kernels for a model, the number of graph connections to make during a lookback period, the maximum depth of a tree in a random forest, a minimum sample split, a maximum number of leaf nodes, a minimum number of leaf nodes, and the like. The cost function can be constructed to measure the difference between the outputs inferred using the prediction models 120 and the ground truth annotated to the samples using the labels.

In some instances, the model trainer 140 can generate weak learner or ensemble models. Initially, the model trainer 140 can create a first model (e.g., a decision tree) from the training data 145a. Generating the first model can involve the model trainer 140 identifying which input variables of the training data 145a can best separate the target variables (e.g., unplanned death or upcoming veterinary request), or more precisely, how easily is it to distinguish the possible target variables if an input variable is split at a certain value. For example, the site an animal subject is located in may have a smaller impact on predicting the probability of a veterinary request than an animal subject's food consumption as cataloged in the clinical observation data, so the food consumption is likely a better predictor. The model trainer 140 may determine splits based on a purity measurement. For example, a gini impurity score, or other purity score, may be determined that measures the likelihood that a data point would, if selected at random, be associated with a given class (with a target label being a veterinary request or an unplanned death in this case). Splits are determined by the value of the variable that provides the purest split. As the first model is made and errors from the first model are noted by a loss function (e.g., logarithmic loss function) of a boosting algorithm, training data which is incorrectly classified is used as input for a subsequent model. The subsequent model is generated from the previous model (e.g., the first model) by trying to reduce the errors from the previous model. To perform a gradient descent procedure, the subsequent model is added that reduces the loss (i.e., follows the gradient of the error). The subsequent model can be parameterized and then the parameters can be modified to move in a direction that reduces the residual loss. Models are added sequentially, each correcting its predecessor, until the training data 145a is predicted perfectly or a maximum number of models (e.g., one hundred) have been added to the ensemble. Essentially, the boosting tries to reduce the bias error which arises when models are not able to identify relevant trends in the data. This happens by evaluating an error between the predicted value of the model and the actual value or ground truth value assigned the training data 145a. The output of each model is added to the output of the other models in an effort to correct or improve the final output of the prediction model 120, which includes all of the ensemble models.

Once the set of model parameters are identified, the prediction model 120 has been trained and the model trainer 140 performs the additional processes of testing or validation using the subset of testing data 145b (testing or validation data set). The testing or validation processes includes iterative operations of inputting utterances from the subset of testing data 145b into the prediction model 120 using a validation technique such as K-Fold Cross-Validation, Leave-one-out Cross-Validation, Leave-one-group-out Cross-Validation, Nested Cross-Validation, or the like to tune the hyperparameters and ultimately find the optimal set of hyperparameters. Once the optimal set of hyperparameters are obtained, a reserved test set from the subset of testing data 145b may be input into the prediction model 120 to obtain output (in this example, a prediction of a veterinary request or an unplanned death), and the output is evaluated versus ground truth entities using correlation techniques such as Bland-Altman method and the Spearman's rank correlation coefficients. Further, performance metrics may be calculated such as the error, accuracy, precision, recall, receiver operating characteristic curve (ROC), etc. The metrics may be used to analyze performance of the prediction model 120 for providing recommendations.

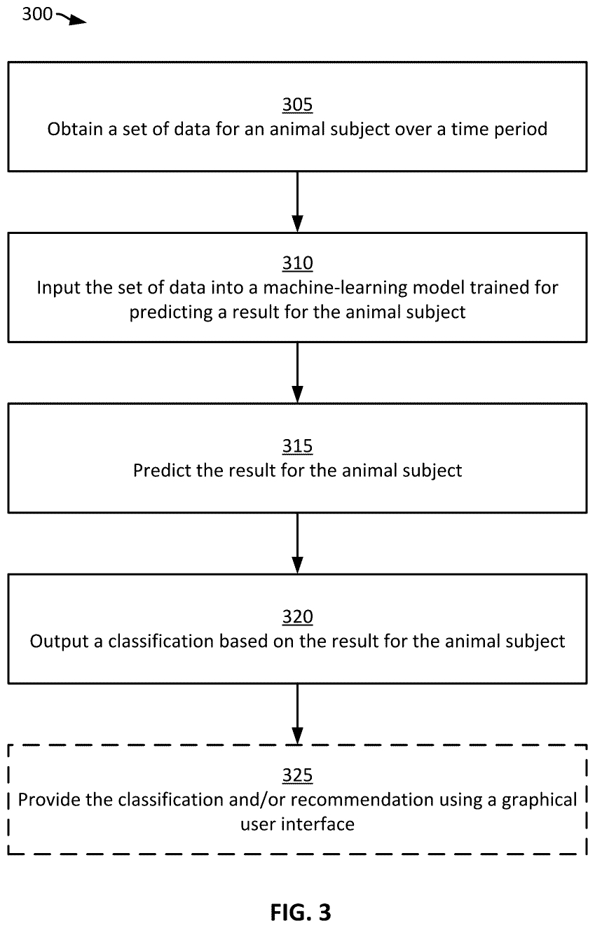

The model training subsystem or service 110 outputs trained models including one or more trained prediction models 160. The one or more trained prediction models 160 may be deployed and used in the implementation subsystem or service 115 for providing predictions 165 to users (as described in detail with respect to FIG. 3). For example, trained prediction models 160 may receive input data 170 including clinical observation data over a time period (e.g., three weeks) for an animal subject, weight measurement data over the time period for the animal subject, outcome status data for the animal subject over the time period, or any combination thereof and provide predictions 165 to a user based on an likelihood of a veterinary request in an upcoming time period (e.g., five days), a treatment required in an upcoming time period (e.g. five days), or an unplanned death for the animal subject. The input data 170 may comprise data stored in the same data structure as used in the data sets 145. For example, the input data 170 is stored in an adjacency graph comprising subgraphs storing clinical observation data over a time period, weight measurement data over the time period, outcome status data for the animal subject over the time period, and/or veterinary treatment record data for the animal subject over the time period.

The implementation subsystem or service 115 comprises deployment tools 175 which are part of the machine learning operationalization framework comprising hardware such as one or more processors (e.g., a CPU, GPU, TPU, FPGA, the like, or any combination thereof), memory, and storage that operates software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands for executing the prediction model 120 in a production environment. In some instances, the deployment tools 175 implement deployment of the prediction model 120 using a cloud platform such as Amazon Web Services (AWS), Google Cloud Platform (GCP), and Microsoft Azure. A cloud platform makes machine learning more accessible, flexible, and cost-effective while allowing developers to build and deploy the prediction model 120 faster.

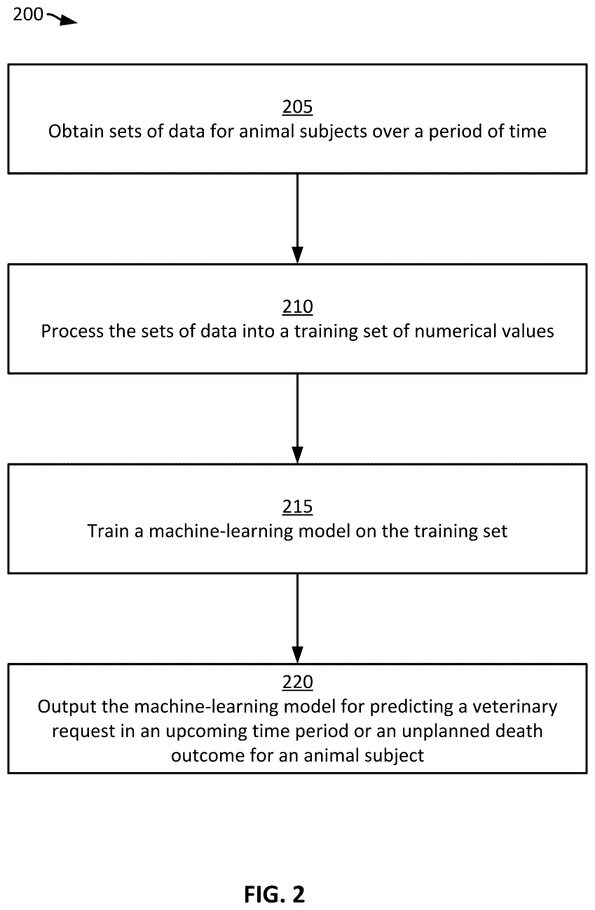

FIG. 2 shows a flowchart illustrating a process 200 for training a machine-learning model according to various embodiments. The processing depicted in FIG. 2 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 2 and described below is intended to be illustrative and non-limiting. Although FIG. 2 depicts the various processing steps occurring in a particular sequence or order, this is not intended to be limiting. In certain alternative embodiments, the steps may be performed in some different order or some steps may also be performed in parallel. In certain embodiments, the processing or a portion of the processing depicted in FIG. 2 may be performed by a computing device such as a computer (e.g., computing device 520 in FIG. 5).

At block 205, sets of data for a plurality of animal subjects are obtained. The sets of data comprise: (i) clinical observation data, (ii) body weight measurement data, (iii) outcome status data, (iv) veterinary treatment record data, or (v) any combination thereof. The clinical observation data, body weight measurement data, veterinary treatment record data, and outcome status data can include free text entries, categorical variable entries, floating point entries, or any combination thereof. In some instances, all of the historical data for the animal subjects are used as inputs for training a machine-learning model. In other instances, a predetermined amount of data (e.g., data falling within a given time frame or window) for an animal subject are selected and used in block 215 as the sets of data input to train the machine-learning model. In certain instances, the predetermined amount of data is all the historical data collected for animal subjects within a look back window (e.g., the three weeks prior to a health event such as requiring veterinary attention or an unplanned death), or absent a health event, all the historical data collected for animal subjects within the most current look back window (e.g., the last three weeks of data). The predetermined amount of data is a tunable hyperparameter (e.g., can be set based on talking with the veterinary staff and how they typically review an animal's history to determine any health problems).

At block 210, the sets of data are processed into a training set of numerical values. Free text entries can be converted into a vector of numerical values using a word embedding vector and categorical variable entries can be converted into numerical values using a mapping between predefined categorical variables and numerical values. The vectors for the free text entries may additionally be reduced in size. Floating point values can remain unchanged.

At block 215, a machine-learning model is trained on the training set. The training can involve generating a first decision tree based on values for the clinical observation data, the body weight measurement data, the veterinary treatment record data, and the outcome status data and then determining an error associated with the first decision tree. A second decision tree can then be generated based on the error and the values for the clinical observation data, the body weight measurement data, the veterinary treatment record data, and the outcome status data. Additional decision trees can be generated until the error reaches an acceptable level or a number of iterations is reached. The machine-learning model includes each of the generated decision trees and is trained for multi-class predictions.

In some instances, the training can involve generating a multi-layer graph neural network. A temporal directed forward-in-time graph may be created for each animal's clinical observations, body weight measurements, and treatment administration data. Information propagates forward over time between observations for each animal and aggregates. The aggregated animal history is then passed through a series of hidden neural network layers, returning a predicted probability of treatment for each animal. The most likely predicted class is subsequently compared against the actual class to calculate an error. Afterward, the learnable parameters can be updated, and another iteration of updates can be performed. This process continues until the model has converged on the best-fit and generalizable (i.e., not overfitting) model.

In some instances, the training can be further optimized based on new training data or expanded training data. For example, more time-point entries corresponding to a training animal subject may be collected through time, and be input to a partially trained model for model optimization. In some instances, the adjacency matrix/graph used to store the training data is advantageous for adding more input entries, as discussed with regard to FIG. 1A.

The machine-learning model ultimately tries to predict 0=health status is all good, 1=vet attention is likely (e.g., the animal subject is sick) in an upcoming time period, 2=the animal subject is likely to experience an unplanned death in an upcoming time period, or 3=a treatment is likely to be administered to the animal subject in an upcoming time period. The likelihood or high probability for a given class refers to the model's confidence in making that prediction. For example, a probability of 70% for a vet attention means the model is 70% confident that the observations point towards a vet attention being needed in the upcoming time period. This is different from classification accuracy which speaks to the actual correctness of that prediction as verified by a person. So, the machine-learning model can be confident (70%) but be wrong (inaccurate). The confidence equals the likelihood or high probability for a given class. The machine-learning model does not make a distinction between when exactly an event will happen just that it will likely happen within an upcoming time period (e.g., a 0-5 day window). This is because the model is trained on the sets of data where the vet requests or attention labels are in the future. Consequently, the training set of numerical values may factor in the observational data from the look back window period and then if a vet request was opened anytime within the upcoming time period (e.g., 0-5 days later) this is labeled as a positive example to learn from (e.g., a vet request being opened may be defined as anytime within the 0-5 day window).

At block 220, the machine-learning model is output for predicting in an inference phase whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period. Currently, veterinary technicians and staff have to painstakingly review all the clinical observations, body weight measurements, and other details from animal by animal to determine which animal they need to observe first. Or the other strategy is to simply go room by room. In either case, if the number of animal subjects is high, the veterinary technicians and staff are unclear about which animals may need further attention until they have completed their assessment. This wastes resources including human resources and, in some instances, may be a highly inaccurate means for assessing the health of the animal subjects. Predictions from a machine-learning can assist in the triage process without the need for veterinary technicians and staff to first review all animal histories and can increase the overall accuracy of the health assessment.

After the machine learning model is trained, a model artifact is created that constitutes the fully trained model. The machine learning model can be used to make predictions on new data by exposing it through some sort of service. This is typically done by loading the machine learning model artifact from disk, storing it in memory on some computerized system, and then creating a REST API endpoint that allows other applications the ability to pass data to the model and the model endpoint returns a class prediction as well as a probability. Making predictions from a trained machine learning model is referred to as inference. Only the clinical observations (Table 1) and body weight tables (Table 2) are required for inference because the goal is to predict the outcomes in advance of them occurring. The clinical observations and body weight tables follow the same pre-processing and merging pipeline described for training.

FIG. 3 is a flowchart illustrating a process 300 for using a machine-learning model to predict an animal health result according to various embodiments. The processing depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different order or some steps may also be performed in parallel. In certain embodiments, the processing or a portion of the processing depicted in FIG. 3 may be performed by a computing device such as a computer (e.g., computing device 520 in FIG. 5).

At block 305, a set of data over a time period is obtained for an animal subject. The set of data may include: (i) clinical observation data, (ii) body weight measurement data, (iii) outcome status data, (iv) veterinary treatment record data, or (v) any combination thereof. The clinical observation data, body weight measurement data, veterinary treatment record data, and outcome status data can include free text entries, categorical variable entries, floating point entries, date-time entries, numeric entries, boolean entries. or any combination thereof. The set of data may be preprocessed before being input to a machine-learning model in block 310. For instance, free text entries can be converted into a vector of numerical values using a word embedding vector and categorical variable entries can be converted into numerical values using a mapping between predefined categorical variables and numerical values. The vectors for the free text entries may additionally be reduced in size using dimensionality techniques, such as principal components. Floating point values, the numeric entries, or boolean entries can remain unchanged. The set of data may be further pre-processed using similar pre-processing techniques disclosed in FIG. 1A, for example, by the data preparation module 130. The set of data, or the pre-processed set of data is stored in a data structure that is appropriate for inputting into a machine-learning model at block 310 (e.g., a list, a graph, a table, a matrix, or the like). For example, the set of data can be stored in an adjacency graph to be used by a multi-layer graph neural network.

At block 310, the set of data is input into a machine-learning model having a learned set of model parameters for predicting a result for the animal subject. In some instances, the machine-learning model is obtained via the processes described with respect to FIGS. 1A and 2. The machine-learning model may comprise an ensemble of classifiers, and the learned set of parameters are associated with relationships computed by a boosting algorithm. In certain instances, the boosting algorithm is an additive gradient boosting algorithm. The machine-learning model may comprise a neural network classifier, and the learned set of parameters are associated with relationships computed by a gradient descent algorithm. In some instances, the neural network classifier is a multi-layer graph neural network. In certain instances, the multi-layer graph neural network is constructed based on temporal, directed forward-in-time graphs. The set of data may be input into the machine-learning model via a graphical user interface (GUI).

At block 315, the result for the animal subject is predicted using the machine-learning model. The machine-learning model may provide a multi-class prediction, where a first result is associated with a first prediction class (e.g., healthy animal subject), a second result is associated with a second prediction class (e.g., veterinary request), a third result is associated with a third prediction class (e.g., unplanned death), and/or a fourth result is associated with a fourth prediction class (e.g., treatment to be administered). In some instances, the result is stored in a data structure that is appropriate for output or display on the GUI.

At block 320, a classification is output based on the result for the animal subject. The classification can involve comparing the result for the animal subject to a determined threshold and classifying the animal subject as having the veterinary request in the upcoming time period or the unplanned death outcome based on the comparison. For example, the result may include a confidence of the prediction and the classification can be based on a comparison between the confidence to the threshold. For example, if the result indicates an 80% confidence that the animal subject will have an upcoming veterinary request and the threshold is 75%, the classification can be that the animal subject is predicted to have the veterinary request. But, if the result indicates a 50% confidence, the classification can be that the animal subject is not predicted to have the veterinary request. Based on the classification a probability of requiring attention or a recommendation for the veterinary request can be provided. Additionally, a recommendation about a course of action may be provided if the classification indicates a likely unplanned death for the animal subject, or a recommendation for a treatment can be provided and the veterinary experts can determine which animal subjects to examine first to assess the proper course of treatment. In some instances, the probabilities and/or recommendation is provided to a user such as a veterinary technician (e.g., a health care worker associated with the animal subject). The classification probabilities and/or recommendation can promote animal health and well-being by consistently and accurately detecting when an animal subject is expected to experience an undesired outcome so that an action can be taken to improve the expected outcome.

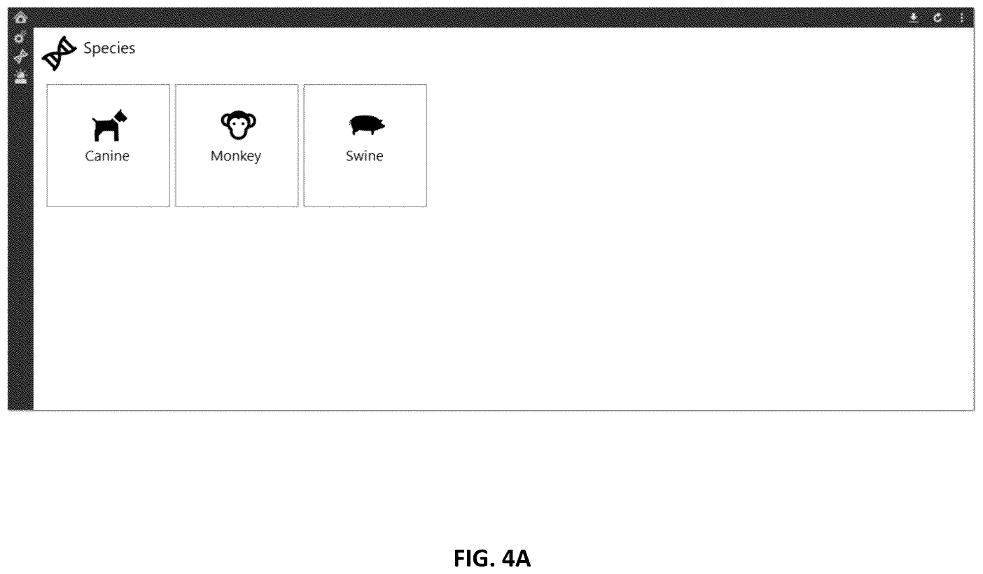

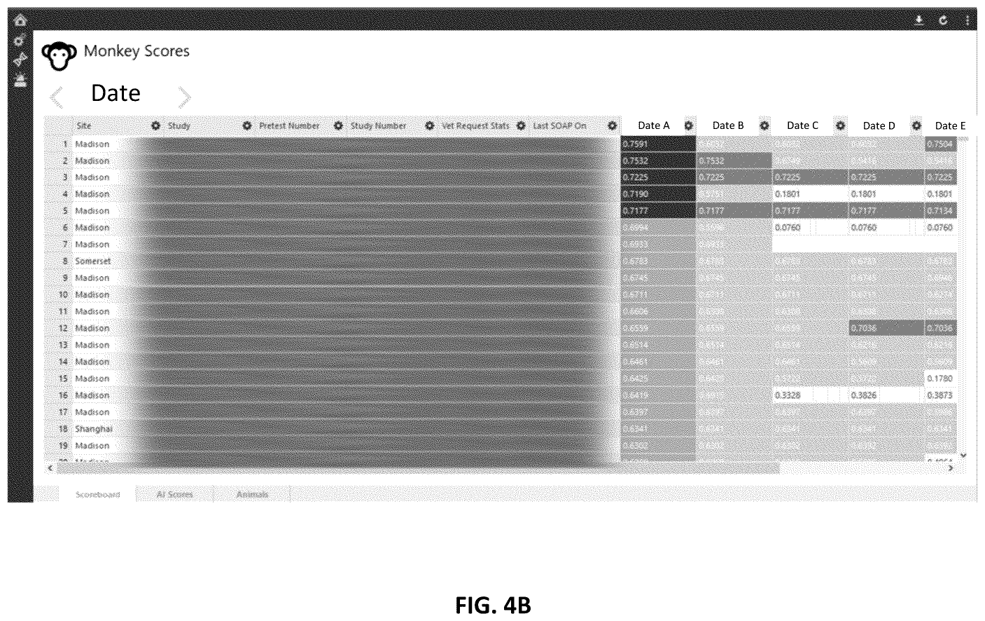

Optionally, at block 325, the classification and/or the recommendation can be provided to users such as veterinary technicians via a graphical user interface (GUI). An exemplary GUI is shown in FIGS. 4A and 4B. As can be seen in FIG. 4A, each icon may represent different species, for example, canine, monkey, and swine. By clicking on each icon, a secondary user interface (UI) may be presented that demonstrate the classification and/or the probability of requiring attention regarding an animal subject. FIG. 4B shows the secondary UI demonstrating the probability of requiring attention regarding a monkey. As shown in FIG. 4B, the probability of an animal likely requiring attention based on the machine learning model classification is being presented. The probability being presented may be associated with the sum of all probabilities associated with non-health classes, such as probability of veterinary request, treatment, or unplanned death in the coming days. Animals with scores exceeding a predetermined threshold may be color coded based on the threshold to make it easier to identify animals that the model has flagged. The historical animal health scores may also be presented for context so that veterinary technicians and staff can identify animal subjects whose health may be deteriorating over time. For example, a dark shaded cell in FIG. 4B represents that the animal subject has a probability greater than 70% of requiring attention, and a light shaded cell representing the probability between 40% and 70%. When the probability is under 40%, the corresponding cell may be not shaded. As shown in FIG. 4B, the secondary UI comprises multiple sheets (e.g., scoreboard, AI scores, and/or animals) and may be easily switched between different sheets based on user needs.

In some instances, a full clinical report for the animal subject may also be provided by the GUI or the secondary UI. The full clinical report may comprise the obtained sets of data for the animal subject at block 305, the predicted result generated by the machine learning model at block 315, and the classification probabilities (and/or recommendation) output at block 320 for the entire animal subject's history. By examining the full report, the veterinary technicians and staff can better triage which animals require attention first based on their specific clinical observations.

In some instances, a notification is displayed using the GUI or a secondary UI. For example, when a user clicks on the animal species icon on the GUI, a secondary UI will display a health score or an AI score associated with an animal subject of the animal species. In some instances, the displayed score or similar notification is color-coded. For example, a red notification may represent that the animal subject requires attention, yellow representing monitoring, and a white background meaning nothing is predicted to be remarkable. The color codes may be predetermined with associated thresholds.

In some instances, a measure of the animal species is also displayed using the GUI. For example, an account or a percentage of monkey subjects that have a health score exceeds a predetermined threshold may be displayed associated with the monkey icon in FIG. 4A. In some instances, when the account or the percentage exceeds a predetermined threshold, a notification to a user may be automatically pushed to the user. The notification may be pushed using the GUI, or via a communication system such as an instance messaging system.

FIG. 5 illustrates a non-limiting example of a computing environment 510 in which various systems, methods, process, and data structures described herein may be implemented. The computing environment 510 is only one example of a suitable computing environment and is not intended to suggest any limitation as to the scope of use or functionality of the systems, methods, and data structures described herein. Neither should computing environment 510 be interpreted as having any dependency or requirement relating to any one or combination of components illustrated in computing environment 510. A subset of systems, methods, and data structures shown in FIG. 5 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment 510 includes a computing device 520 (e.g., a computer or other type of machines such as sequencers, photo cells, photo multiplier tubes, optical readers, sensors, etc.), including a processing unit 521, a system memory 522, and a system bus 523 that operatively couples various system components including the system memory 522 to the processing unit 521. There may be only one or there may be more than one processing unit 521, such that the processor of computing device 520 includes a single central-processing unit (CPU), or a plurality of processing units, commonly referred to as a parallel processing environment. The computing device 520 may be a conventional computer, a distributed computer, or any other type of computer, which may include a graphical processing unit (GPU) used for computation.

The system bus 523 may be any of several types of bus structures including a memory bus or memory controller, a peripheral bus, and a local bus using any of a variety of bus architectures. The system memory 522 may also be referred to as simply the memory, and includes read only memory (ROM) 524 and random access memory (RAM) 525. A basic input/output system (BIOS) 526, containing the basic routines that help to transfer information between elements within the computing device 520, such as during start-up, is stored in ROM 524. The computing device 520 may further include a hard disk drive interface 527 for reading from and writing to a hard disk, not shown, a magnetic disk drive 528 for reading from or writing to a removable magnetic disk 529, and an optical disk drive 530 for reading from or writing to a removable optical disk 531 such as a CD ROM or other optical media.

The hard disk drive, magnetic disk drive 528, and optical disk drive 530 are connected to the system bus 523 by a hard disk drive interface 532, a magnetic disk drive interface 533, and an optical disk drive interface 534, respectively. The drives and their associated computer-readable media provide nonvolatile storage of computer-readable instructions, data structures, program modules and other data for the computing device 520. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 529, optical disk 531, ROM 524, or RAM 525, including an operating system 535, one or more application programs 536, other program modules 537, and program data 538. A user may enter commands and information into the computing device 520 through input devices such as a keyboard 540 and pointing device 542. Other input devices (not shown) may include a microphone, joystick, game pad, satellite dish, scanner, or the like. These and other input devices are often connected to the processing unit 521 through a serial port interface 546 that is coupled to the system bus, but may be connected by other interfaces, such as a parallel port, game port, or a universal serial bus (USB). A monitor 547 or other type of display device is also connected to the system bus 523 via an interface, such as a video adapter 548. In addition to the monitor, computers typically include other peripheral output devices (not shown), such as speakers and printers.

The computing device 520 may operate in a networked environment using logical connections to one or more remote computers, such as remote computer 549. These logical connections may be achieved by a communication device coupled to or a part of the computing device 520, or in other manners. The remote computer 549 may be another computer, a server, a router, a network PC, a client, a peer device or other common network node, and typically includes many or all of the elements described above relative to the computing device 520. The logical connections depicted in FIG. 5 include a local-area network (LAN) 551 and a wide-area network (WAN) 552. Such networking environments are commonplace in office networks, enterprise-wide computer networks, intranets and the Internet, which all are types of networks.

When used in a LAN-networking environment, the computing device 520 is connected to the local-area network 551 through a network interface or adapter 553, which is one type of communications device. When used in a WAN-networking environment, the computing device 520 often includes a modem 554, a type of communications device, or any other type of communications device for establishing communications over the wide-area network 552. The modem 554, which may be internal or external, is connected to the system bus 523 via the serial port interface 546. In a networked environment, program modules, such as application programs 536 depicted relative to the computing device 520, or portions thereof, may be stored in the remote memory storage device. It is appreciated that the network connections shown are non-limiting examples and other communications devices for establishing a communications link between computers may be used.

III. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A method comprising:
obtaining sets of data for a plurality of animal subjects over a time period, wherein the sets of data comprise: (i) clinical observation data, (ii) body weight measurement data, (iii) outcome status data, (iv) veterinary treatment record data, or (v) any combination thereof;
processing the sets of data into a training set of numerical values;
training a machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and
outputting the machine-learning model.

2. The method of claim 1, wherein processing the sets of data into the training set of numerical values comprises:
(i) (a) determining a free text entry in the sets of data;
(b) applying an embedding model to the free text entry to generate a vector of the free text entry;
(c) reducing a size of the vector using a principal component analysis reduction method; and
(d) including the vector in the training set; or
(ii) (a) determining a categorical variable entry in the sets of data;
(b) converting the categorical variable entry into a numerical value using a mapping between numerical values and categorical variable entries; and
(c) including the numerical value in the training set.

3. The method of claim 1, further comprising, prior to training the machine-learning model:
labelling the numerical values of the training set with an unplanned death indicator, a veterinary request indicator, or a veterinary treatment indicator.

4. The method of claim 3, wherein labelling the numerical values of the training set comprises:
(i) determining the clinical observation data for an animal subject of the plurality of animal subjects includes the veterinary request indicator and labelling the training set with the veterinary request indicator for the animal subject;
(ii) determining the outcome status data for an animal subject of the plurality of animal subjects includes the unplanned death indicator and labelling the training set with the unplanned death indicator for the animal subject;
(iii) determining the veterinary treatment record data for an animal subject of the plurality of animal subjects includes the veterinary treatment indicator and labelling the training set with the veterinary treatment indicator for the animal subject; or
(iv) any combination of (i)-(iii).

5. The method of claim 1, wherein training the machine-learning model comprises:
(i) (a) generating a first decision tree based on values for the clinical observation data, the body weight measurement data, the veterinary treatment record data, and the outcome status data;
(b) determining an error associated with the first decision tree; and
(c) generating a second decision tree based on the error and the values for the clinical observation data, the body weight measurement data, the veterinary treatment record data, and the outcome status data, wherein the machine-learning model includes the first decision tree and the second decision tree; or
(ii) (a) generating a chronologically ordered temporal graph for each animal subject based on the values for the clinical observation data, the body weight measurements data, the veterinary treatment record data, and the outcome status data;
(b) transforming the chronologically ordered temporal graph to a pre-processed table for classification; and
(c) automatically adjusting weights based on a predetermined condition.

6. The method of claim 1, wherein the machine-learning model comprises an additive gradient boosting algorithm or a multi-layer graph neural network algorithm.

7. A method comprising:
obtaining a set of data for an animal subject over a time period, the set of data including: (i) clinical observation data, (ii) body weight measurement data, (iii) outcome status data, (iv) veterinary treatment record data, or (v) any combination thereof;
inputting the set of data into a machine-learning model trained for predicting a result for the animal subject, wherein the result comprises a veterinary request in an upcoming time period, an unplanned death outcome for the animal subject, or a treatment is likely to be administered to the animal subject in an upcoming time period;
predicting, using the machine-learning model, the result for the animal subject; and
outputting a classification based on the result for the animal subject.

8. The method of claim 7, wherein the machine-learning model is an additive gradient boosting algorithm or a multi-layer graph neural network algorithm.

9. The method of claim 7, wherein the classification comprises comparing the result for the animal subject to a determined threshold and classifying the animal subject as having the veterinary request in the upcoming time period, the unplanned death outcome for the animal subject is likely in an upcoming time period, or a treatment to the animal subject to be administered in an upcoming time period based on the comparison.

10. The method of claim 9, further comprising providing a recommendation based on the classification of the animal subject.

11. The method of claim 10, further comprising providing the classification and/or the recommendation to a user through a graphical user interface (GUI).

12. The method of claim 7, further comprising, prior to receiving the set of data for the animal subject:
obtaining sets of data for a plurality of animal subjects over a time period, wherein the sets of data comprise the clinical observation data, the body weight measurement data, the outcome status data, the veterinary treatment record data, or any combination thereof;
processing the sets of data into a training set of numerical values;
training the machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and
outputting the machine-learning model.

13. The method of claim 12, wherein training the machine-learning model comprises:
(i) (a) generating a first decision tree based on values for the clinical observation data, the body weight measurement data, the veterinary treatment record data, and the outcome status data;
(b) determining an error associated with the first decision tree; and
(c) generating a second decision tree based on the error and the values for the clinical observation data, the body weight measurement data, the veterinary treatment record data, and the outcome status data, wherein the machine-learning model includes the first decision tree and the second decision tree; or
(ii) (a) generating a chronologically ordered temporal graph for each animal subject based on the values for the clinical observation data, the body weight measurements data, the veterinary treatment record data, and the outcome status data;
(b) transforming the chronologically ordered temporal graph to a pre-processed table for classification; and
(c) automatically adjusting weights based on a predetermined condition.

14. A system comprising:
one or more data processors; and
a non-transitory computer readable storage medium containing instructions which, when executed on the one or more data processors, cause the one or more data processors to perform:
obtaining sets of data for a plurality of animal subjects over a time period, wherein the sets of data comprise: (i) clinical observation data, (ii) body weight measurement data, (iii) outcome status data, (iv) veterinary treatment record data, or (v) any combination thereof;
processing the sets of data into a training set of numerical values;
training a machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and
outputting the machine-learning model.

15. The system of claim 14, wherein processing the sets of data into the training set of numerical values comprises:
(i) (a) determining a free text entry in the sets of data;
(b) applying an embedding model to the free text entry to generate a vector of the free text entry;
(c) reducing a size of the vector using a principal component analysis reduction method; and
(d) including the vector in the training set; or
(ii) (a) determining a categorical variable entry in the sets of data;
(b) converting the categorical variable entry into a numerical value using a mapping between numerical values and categorical variable entries; and
(c) including the numerical value in the training set.

16. The system of claim 14, wherein the one or more data processors are further caused to perform, prior to training the machine-learning model:
labelling the numerical values of the training set with an unplanned death indicator, a veterinary request indicator, or a veterinary treatment indicator, wherein labelling the numerical values of the training set comprises:
(i) determining the clinical observation data for an animal subject of the plurality of animal subjects includes the veterinary request indicator and labelling the training set with the veterinary request indicator for the animal subject;
(ii) determining the outcome status data for an animal subject of the plurality of animal subjects includes the unplanned death indicator and labelling the training set with the unplanned death indicator for the animal subject;
(iii) determining the veterinary treatment record data for an animal subject of the plurality of animal subjects includes the veterinary treatment indicator and labelling the training set with the veterinary treatment indicator for the animal subject; or
(iv) any combination of (i)-(iii).

17. The system of claim 14, wherein training the machine-learning model comprises:
(i) (a) generating a first decision tree based on values for the clinical observation data, the body weight measurement data, the veterinary treatment record data, and the outcome status data;
(b) determining an error associated with the first decision tree; and
(c) generating a second decision tree based on the error and the values for the clinical observation data, the body weight measurement data, the veterinary treatment record data, and the outcome status data, wherein the machine-learning model includes the first decision tree and the second decision tree; or
(ii) (a) generating a chronologically ordered temporal graph for each animal subject based on the values for the clinical observation data, the body weight measurements data, the veterinary treatment record data, and the outcome status data;
(b) transforming the chronologically ordered temporal graph to a pre-processed table for classification; and
(c) automatically adjusting weights based on a predetermined condition.

18. A system comprising:
one or more data processors; and
a non-transitory computer readable storage medium containing instructions which, when executed on the one or more data processors, cause the one or more data processors to perform:
obtaining a set of data for an animal subject over a time period, the set of data including: (i) clinical observation data, (ii) body weight measurement data, (iii) outcome status data, (iv) veterinary treatment record data, or (v) any combination thereof;
inputting the set of data into a machine-learning model trained for predicting a result for the animal subject, wherein the result comprises a veterinary request in an upcoming time period, an unplanned death outcome for the animal subject, or a treatment is likely to be administered to the animal subject in an upcoming time period;
predicting, using the machine-learning model, the result for the animal subject; and
outputting a classification based on the result for the animal subject.

19. The system of claim 18, wherein the classification comprises comparing the result for the animal subject to a determined threshold and classifying the animal subject as having the veterinary request in the upcoming time period, the unplanned death outcome for the animal subject is likely in an upcoming time period, or a treatment to the animal subject to be administered in an upcoming time period based on the comparison.

20. The system of claim 18, wherein the one or more data processors are further caused to perform, prior to receiving the set of data for the animal subject:
obtaining sets of data for a plurality of animal subjects over a time period, wherein the sets of data comprise the clinical observation data, the body weight measurement data, the outcome status data, the veterinary treatment record data, or any combination thereof;
processing the sets of data into a training set of numerical values;
training the machine-learning model on the training set to predict whether health of an animal subject is normal, veterinary attention for the animal subject is likely required in an upcoming time period, an unplanned death outcome for the animal subject is likely in an upcoming time period, or a treatment is likely to be administered to the animal subject in an upcoming time period; and
outputting the machine-learning model.
